(19) United States
(12) Patent Application Publication
(10) Pub. No.: US 2004/0171517 A1
(43) Pub. Date: Sep. 2, 2004

(54) Compounds and methods for modulating bacterial functions

(76) Inventors: Tony Romeo, Decatur, GA (US); Xin Wang, Atlanta, GA (US); Kazushi Suzuki, Decatur, GA (US)

Correspondence Address: MERCHANT & GOULD PC, P.O. BOX 2903, MINNEAPOLIS, MN, 55402-0903, US

(21) Appl. No.: 10/675,738

(22) Filed: Sep. 29, 2003

Related U.S. Application Data
(60) Provisional application No. 60/414,351, filed on Sep. 30, 2002.

(51) Int. Cl. A61K 38/00; C12N 1/21

(52) U.S. Cl. 514/2; 435/252.33

(57) ABSTRACT

There is provided a method of reducing biofilm formation by biofilm-forming bacterial strains such as E. coli, Salmonella, Klebsiella, or a related gamma proteobacteria. CsrA activity levels in the cell may be modulated to impact biofilm formation and glycogen metabolism. CsrA levels may be modulated by modulating the expression of csrB RNA and UvrY and BarA gene products. By increasing levels of CsrB, BarA or SdiA in a bacterial cell or increasing the rate of UvrY phosphorylation, one can increase the levels of active UvrY in the strain. There is also provided a modulator of CsrA activity in a bacteria comprising a nucleotide sequence containing the sequence element CAGGAUG.

### CROSS-REFERENCE TO RELATED APPLICATION

This application claims priority from U.S. provisional application No. 60/414,351, filed Sep. 30, 2002, which is pending.

### FIELD OF THE INVENTION

The invention relates to the modulation of bacterial functions and particularly to modulators of functions in biofilm producing bacteria.

### BACKGROUND OF THE INVENTION

To persist in nature, bacteria must be able to compete and survive under varying growth conditions. To accomplish this task, they possess regulatory systems that permit them to recognize and adapt to a changing environment. In Escherichia coli and related species, the transition from exponential growth to stationary phase growth is accompanied by striking physiological changes, which produce cells that are more stress resistant, slower metabolizing, and better at scavenging nutrients. These adaptations are brought about largely through changes in gene expression that are coordinated through global regulatory networks.

Thus, it is an object of the invention to provide compounds and methods for modulating bacterial functions.

### SUMMARY OF THE INVENTION

The present invention discloses interactions among different types of global regulatory systems that affect stationary phase gene expression, and uses and methods for modulating bacterial functions employing these interactions.

In an embodiment of the invention there is provided a modulator of biofilm formation in biofilm-producing bacteria.

In an embodiment of the invention there is provided a method of modulating biofilm formation by biofilm-producing bacteria.

### BRIEF DESCRIPTION OF THE DRAWINGS

FIG. 1 is a graphical representation of the results of northern analysis of CsrB levels in E. coli K-12 MG1655 and isogenic barA and uvrY knockout mutants. RNA from cultures harvested at 2 h post-exponential phase of growth was probed for the CsrB transcript. Panel A depicts a blot representative of the observed results. An overexposed film is shown to reveal CsrB RNA from the uvrY mutant. Panel B shows quantification of the signal by phosphorimage analysis. Signals were normalized with respect to MG1655, which was assigned a value of 100%. Each bar represents the mean value obtained from 2 independent experiments. Where error bars depicting the standard deviations (SD) are not apparent, the SD were below the resolution of the graph.

FIG. 2 is a graphical representation of the effects of the uvrY or barA null mutations on expression from csrA‘-’lacZ translational or csrB-lacZ transcriptional fusions. β-Galactosidase specific activities expressed from csrA‘-’lacZ in strains KSA712 (wild type), UY KSA712 (uvrY::cam), and BA KSA712 (barA::kanR) (A), and from csrB-lacZ in strains KSB837 (wild type), UY KSB837 (uvrY::cam), or BA KSB837 (barA::kanR) (B) are shown. β-Galactosidase activities in the wild type, uvrY::cam, or barA::kanR strains are shown as closed circles, squares, or triangles, respectively. Growth (A600) of the respective strains is depicted by open circles, squares, and triangles.

FIG. 3 is a graphical representation of the effects of csrA and uvrY mutations on expression from a barA-lacZ transcriptional fusion. (A) β-Galactosidase specific activities expressed from a barA-lacZ fusion in strains CAG HS703 (wild type) and TR1-5 CAG HS703 (csrA::kanR) are shown as closed or open circles, respectively. (B) Activities in strains HS703 and UY HS703 (uvrY::cam) are shown as closed or open circles, respectively. In each panel, growth (A600) of the respective strains is depicted by closed or open squares.

FIG. 4 is a graphical representation of the results of complementation studies: effects of ectopic expression of csrA (pCRA16), uvrY (pUY14) or barA (pBA29) on expression of a csrB-lacZ transcriptional fusion in isogenic csrA, uvrY, or barA mutants of KSB837. The vector control was pBR322 in each case. Specific β-galactosidase activities and growth (A600) at 24 h are shown as bars and closed circles, respectively. This experiment was repeated with essentially identical results.

FIG. 5 is a graphical representation of the effects of the csrB or uvrY null mutations and uvrY overexpression on a chromosomal glgCA‘-’lacZ translational fusion. (A) β-Galactosidase activities expressed from glgCA‘-’lacZ in strains KSGA18 (closed circles) and UY KSGA18 (uvrY::cam) (open circles), (B) KSGA18[pBR322] (closed circles) and KSGA18[pUY14] (open circles), (C) RG KSGA18[pBR322] (csrB::cam) (closed circles) and RG KSGA18[pUY14] (csrB::cam) (open circles) (C) are shown. In each panel, growth (A600) of the respective strains is depicted by closed or open squares.

FIG. 6 is a pictorial (A) and graphical (B) representation of the results of an in vitro transcription-translation of the csrB-lacZ transcriptional fusion carried on pCBZ1. Reaction mixtures contained pCBZ1 (csrB-lacZ) or vector only (1.6 μg), as indicated. Reactions were conducted in the absence or presence of UvrY protein, in an S-30 extract from UY CF7789 (uvrY::cam). (A) Labelled proteins were analyzed by SDS-PAGE and fluorography. The position of an unlabeled standard of β-galactosidase (LacZ) is shown. (B) Incorporation of [³⁵S]-methionine into the LacZ polypeptide was determined by liquid scintillation counting. Approximately 5.5×10³ cpm is equivalent to 1 pmol of LacZ polypeptide per reaction per h.

FIG. 7 is a graphical representation of the effects of sdiA disruption and overexpression on expression of chromosomal uvrY‘-’lacZ and csrA‘-’lacZ translational fusions and a csrB-lacZ transcriptional fusion. Expression from csrA‘-’lacZ in (A) KSA712 and SA KSA712 (sdiA::kanR) and (B) KSA712[pBR322] and KSA712[pSdiA]. Expression from csrB-lacZ in strains (C) KSB837 and SA KSB837 (sdiA::kanR) and (D) KSB837[pBR322] and KSB837[pSdiA]. Expression of uvrY‘-’lacZ in strains (E) KSY009 and SA KSY009 (sdiA::kanR) and (F) KSYOO9[pBR322] and KSY009[pSdiA]. In each panel, β-Galactosidase activities in the wild type strain are shown as closed circles, whereas activities in sdiA::kanR or sdiA overexpressing strains are shown as open circles. Growth (A600) of the respective strains is depicted by closed or open squares. This experiment was repeated in its entirety, with essentially identical results.

FIG. 8 is a graphical representation of the effects of csrB, uvry, barA and sdiA null mutations and ectopic expression of uvrY, barA, and sdiA on 24 h biofilms grown in microtiter wells. (A) Biofilm formation by the parent strain MG1655 and isogenic mutants, as indicated. (B) Effects of increased gene dosage of uvrY (pUY14), barA (pBA29), sdiA (pSdiA) on biofilm formation by MG1655. (C) Same as (B), except in a csrB null mutant (RG1-B MG1655). Bars show the average and standard error of three experiments, with three samples per experiment. Double asterisks indicate statistically significant differences between strains of a given set (P<0.01).

FIG. 9 is a schematic diagram depicting a summary of the regulatory interactions of CsrA/B, BarA/UvrY and SdiA. CsrA activates csrB transcription indirectly (20). This effect of CsrA requires functional UvrY, which directly activates csrB transcription. The effect of CsrA on csrB is mediated in part by activation of barA expression, but apparently also involves a BarA-independent, UvrY-dependent mechanism, shown as [X]. UvrY also activates the expression of barA, in an autoregulatory loop. SdiA activates the expression of uvrY, and to a lesser extent, that of csrB. Finally, CsrB RNA binds to ˜18 subunits of CsrA protein and antagonizes its regulatory effects in the cell (31, 45).

FIG. 10 is a depiction of the RNA sequence of the g/g leader toeprint, structure mapping and footprint results. The positions of the CsrA toeprint, as well as the glgC SD sequence and start codon (Met), are shown in bold type. Positions of cleavage by the single-strand specific probes RNase T1 (G specific) and Pb²⁺ in the absence of bound CsrA are indicated by filled arrowheads and filled circles respectively. The structure of the hairpin loop in the absence of bound CsrA is shown. Nucleotides in which bound CsrA increases (+) or decreases (−) cleavage are indicated. Numbering is from the start of glgCAP transcription.

### DETAILED DESCRIPTION OF THE INVENTION

The global regulatory system Csr (carbon storage regulator) represses a variety of stationary phase genes. The central component of this system, CsrA, is a 61 amino acid RNA binding protein. This protein inhibits glycogen biosynthesis and catabolism, gluconeogenesis, and biofilm formation, whereas it activates glycolysis, acetate metabolism, motility and flagellum biosynthesis in E. coli. The dramatic effect of CsrA on biofilm formation is mediated primarily through its regulatory role in directing glycogen biosynthesis and catabolism. Homologues of csrA exhibit a broad phylogenetic distribution in eubacteria and repress stationary phase genes of Pseudomonas fluorescens, genes involved in plant pathogenesis in Erwinia carotovora, and regulate genes involved in mucosal invasion by Salmonella enterica. The genome of E. coli K-12 was reported by Blattner (1997, ref. 7)

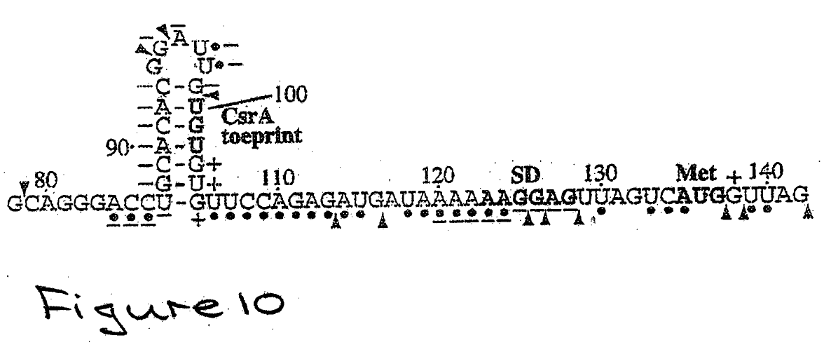

CsrA is capable of post-transcriptional repression or activation, depending upon the particular RNA target. CsrA binds to the untranslated leader of the glgCAP transcript, which encodes enzymes required for glycogen synthesis, at a site that overlaps the glgC Shine-Dalgarno sequence and a second site within a hairpin that is located upstream of the Shine-Dalgarno sequence from nucleotides −52 to +34 (relative to the glgC initiation codon), as shown in FIG. 10. Thus, CsrA blocks ribosome binding and inhibits the initiation of glgC translation. Inhibition of translation is believed to contribute to the observed destabilization of glgCAP mRNA by CsrA. CsrA positively regulates motility in E. coli by binding to and stabilizing the flhDC transcript, which encodes the subunits of a tetrameric DNA-binding protein (FlhD₂C₂) that activates the expression of genes involved in flagellum biosynthesis, motility, and chemotaxis. CsrA binds to the untranslated leader sequence of the flhDC transcript (see also Wei et al., Mol. Microbiol. 40(1): 245-56 (2001).

A second component of Csr is the 366 nucleotide untranslated csrB RNA, which binds to ˜18 CsrA subunits, forming a large globular ribonucleoprotein complex. In vitro transcription-translation studies of glgCAP expression and in vivo csrB disruption and overexpression studies have revealed that csrB RNA functions as an antagonist of CsrA, apparently by sequestering this protein. A highly repeated sequence element that is located in the loops of predicted csrB hairpins, and is related to the sequences involved in glgCAP recognition sites, is believed to mediate the binding of CsrA to csrB. The consensus sequence for the imperfect repeat is CAGGAUG. (See also Liu et al., J. Biol. Chem., 272 (28): 17502-10 (1997)).

The function of csrB RNA as an antagonist of an mRNA binding global regulatory protein offers a novel paradigm for post-transcriptional control by procaryotic regulatory RNA molecules. CsrA indirectly activates csrB transcription, indicative of an autoregulatory mechanism that determines the intracellular activity of CsrA without affecting its level.

HCN biosynthesis (hcnABC) and extracellular protease (aprA) in P. fluorescens CHA0 are regulated indirectly by GacA, a homologue of UvrY, via a post-transcriptional mechanism involving RsmA and RsmZ. PrrB RNA, a 132-nucleotide transcript in P. fluorescens F113 is itself regulated by GacS/GacA. The barA gene of Salmonella enterica positively affects the expression of hilA, which encodes a regulator of genes of pathogenicity island 1 (SPI1). Mutations in csrA or csrB also affect hilA expression. Furthermore, gacS and gacA (alternatively called expS and expA) of Erwinia carotovora affect levels of RsmB.

The sensor-kinase BarA of E. coli was identified as a multicopy suppressor of an envZ defect in the expression of outer membrane proteins, and was shown to activate the transcription of rpoS, which encodes the stationary phase sigma factor RpoS or sigma^{-s}. BarA is a member of the subclass of tripartite sensor kinases. These proteins consist of an N-terminal cytosolic domain, a canonical pair of transmembrane regions linked by a periplasmic bridge, a transmitter domain containing a conserved histidine residue, a central receiver domain with a conserved aspartate residue, and a C-terminal phosphotransfer domain with a conserved histidine residue. Tripartite kinases catalyze the phosphorylation of their cognate response regulators via an ATP-His-Asp-His-Asp phosphorelay. The barA gene appears to play a role in the virulence of uropathogenic E. coli in the urinary tract. BarA is a cognate kinase of UvrY, a response regulator of the FixJ family. The uvrY gene is co-transcribed with uvrC, which encodes a DNA repair enzyme, although UvrY plays no apparent role in DNA repair.

Increased sdiA gene dosage causes uvrY transcript levels to increase by about 10-fold. The sdiA gene encodes a protein of the LuxR family, the members of which contain one domain for binding N-acylated homoserine lactones (AHL) and a second domain for binding DNA. These proteins permit the bacterium to sense and respond to the resident microbial population by binding to AHL beyond a threshold concentration and activating or repressing the transcription of target genes, i.e. they mediate “quorum sensing”. E. coli and Salmonella are not known to synthesize AHL and there are no apparent AHL synthase genes in their genomes.

Regulatory interactions of BarA/UvrY, CsrA/CsrB and SdiA of E. coli are disclosed herein. The UvrY response regulator of E. coli directly activates transcription of csrB and mediates the indirect effects of CsrA on csrB. The BarA sensor-kinase and the DNA binding protein SdiA also regulate csrB transcription, apparently through effects on UvrY phosphorylation and uvrY expression, respectively. Autoregulatory loops characterize these systems. CsrA activates transcription of its RNA antagonist, CsrB, and UvrY stimulates expression of barA, which encodes its cognate sensor-kinase.

The RNA binding protein CsrA and the untranslated RNA CsrB constitute a post-transcriptional regulatory system that has profound effects on central carbon metabolism, motility and multicellular behaviour of E. coli. CsrA is autoregulatory, and indirectly activates transcription of the gene for its RNA antagonist, CsrB. The simplest explanation for this role of CsrA is that CsrA activates a transcriptional activator which the present invention is not limited to any particular mechanism, the results disclosed herein support this hypothesis, and delineate signalling circuitry by which CsrA activates csrB transcription, namely the BarA/UvrY two component signal transduction system.

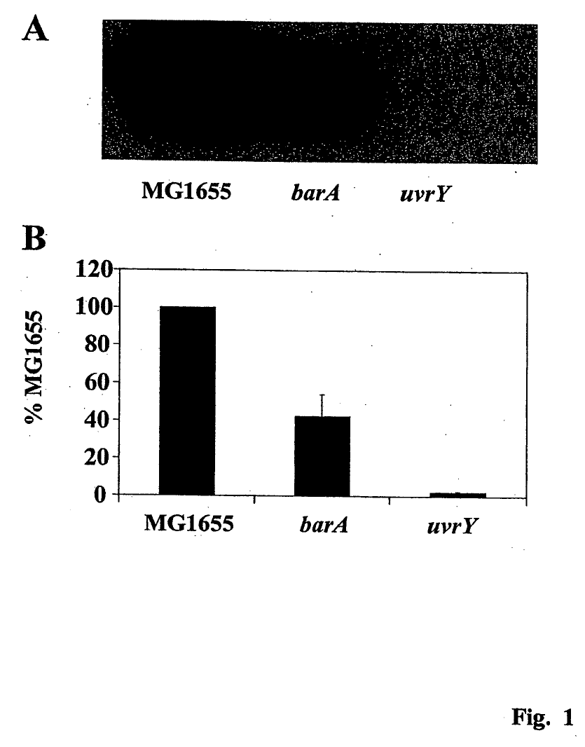

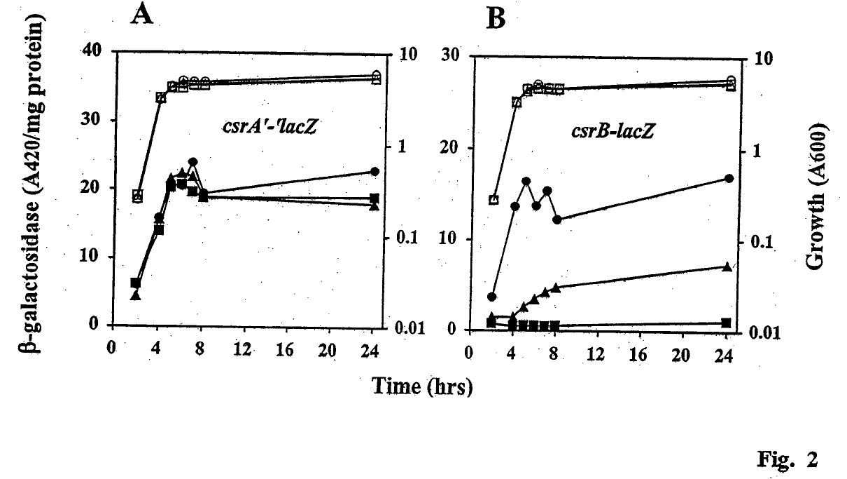

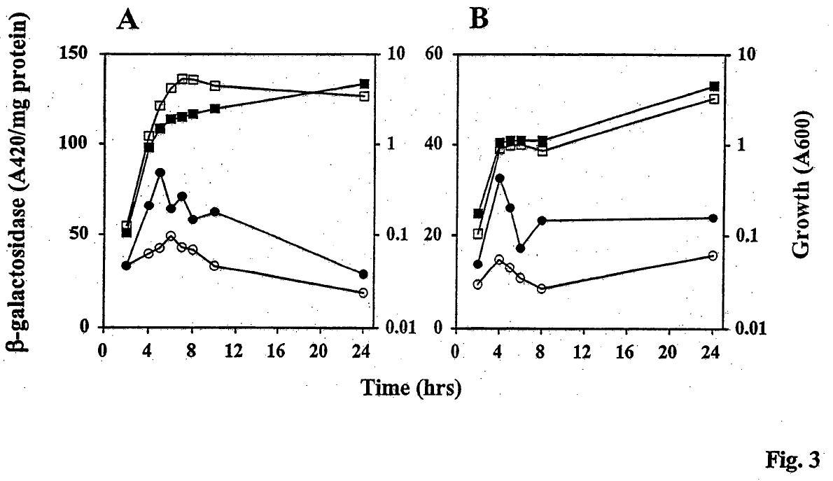

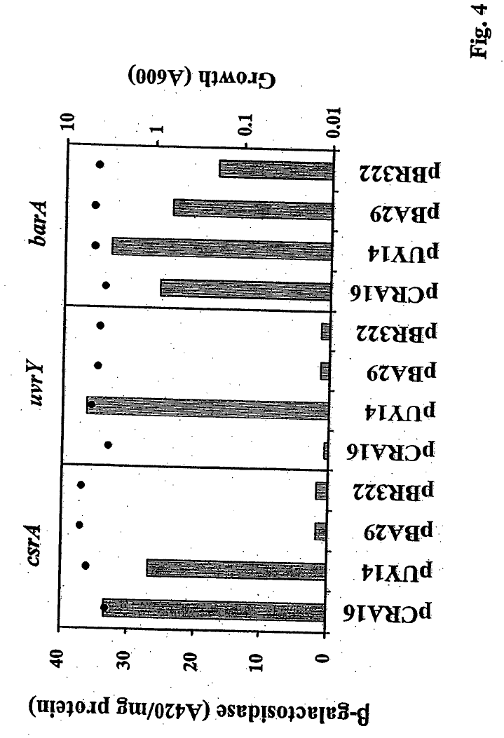

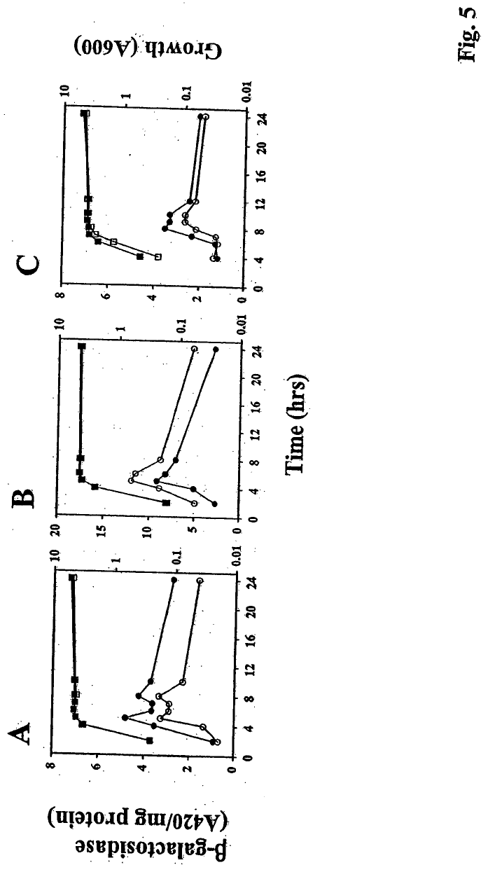

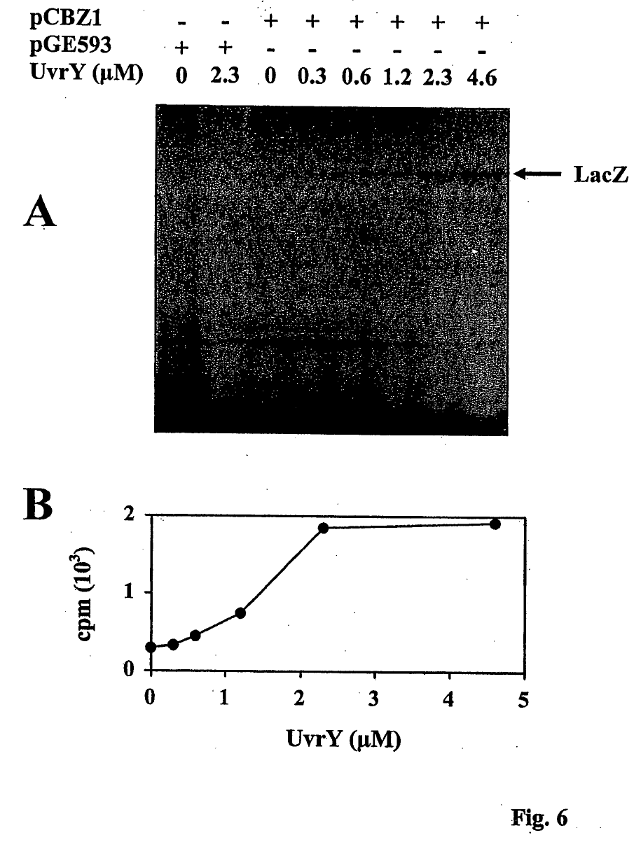

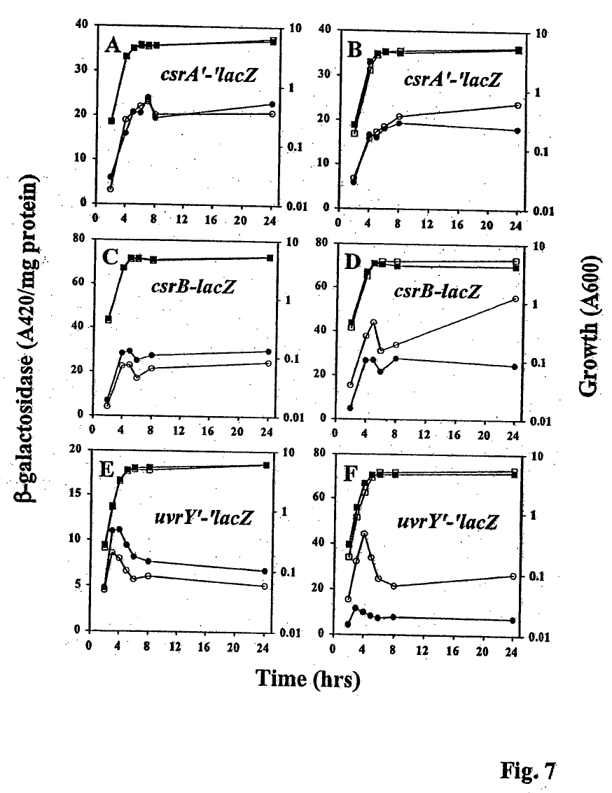

The organization of the signalling circuitry that connects the CsrA/CsrB and BarA/UvrY regulatory systems was defined by several kinds of evidence. First, the steady state levels of CsrB RNA were extremely deficient in csrA (20) and uvrY mutants (FIG. 1). The in vivo expression of a csrB-lacZ transcriptional fusion containing the region from −242 to +4 bp of csrB, relative to the start of transcription, is also highly dependent upon csrA (20) and uvrY (FIG. 2). Since this fusion is capable of expressing only the first 4 nucleotides of the natural csrB transcript, the latter experiment establishes that UvrY activates csrB transcript initiation, and does not only stabilize CsrB RNA. A barA mutant is partially defective for CsrB accumulation and csrB-lacZ expression (FIGS. 1, 2), but this effect of barA is considerably less severe than those of csrA (20) or uvrY mutations (FIGS. 1, 2). Second, barA, uvrY, and csrA itself have no effects on the expression of csrA (FIG. 2). CsrA and UvrY each stimulate barA-lacZ expression ˜1.5 to 2-fold (FIG. 3). Mutations in csrA, barA or uvrY itself did not significantly affect uvrY‘-’lacZ expression. Third, complementation studies with multicopy plasmids showed that uvrY suppresses the defects in csrB-lacZ expression that are caused by csrA, barA or uvrY mutations (FIG. 4). A csrA plasmid clone suppresses the defects of csrA or barA mutants, but has no effect in a uvrY mutant (FIG. 4). Finally, a plasmid clone of barA suppresses the barA defect, but does not affect csrB-lacZ expression in strains defective for uvrY or csrA (FIG. 4). Because ectopic expression of csrA has no effect on csrB if uvrY is defective, while ectopic expression of uvrY suppresses a csrA defect, these complementation experiments provide strong genetic evidence that the effects of CsrA on csrB transcription are mediated through UvrY. CsrA does not appear to affect csrB by regulating the expression of the uvrY gene, although it is a modest activator of the expression of its cognate kinase, BarA (FIG. 3). Fourth, purified CsrA protein failed to regulate csrB-lacZ expression in S-30 transcription-translation assays, indicating that CsrA indirectly activates csrB expression, while recombinant UvrY protein activated the same csrB-lacZ fusion ˜6-fold (FIG. 6). The latter result is believed to represent the first biochemical evidence that UvrY or any of its homologues directly activates gene expression, and positions UvrY immediately upstream from csrB in a signalling pathway. Fifth, mutations in uvrY or barA result in a reduction of the expression of glgC‘-’lacZ and glgCA‘-’lacZ translational fusions (FIG. 5), which are repressed by CsrA and activated by CsrB. While these effects of barA and uvrY were modest, they were in agreement with the modest effects noted for CsrB. The relatively weaker effects of CsrB in comparison to those of CsrA have been observed thus far for all Csr-regulated genes. They are consistent with the finding that CsrB levels in the cell are sufficient to bind only ˜30% of the CsrA protein, assuming full occupation of the ˜18 CsrA-binding sites on CsrB. The relatively lower level of CsrB in the cell relative to CsrA may also account for the modest effects of BarA/UvrY, since UvrY effects on glgCA‘-’lacZ were largely dependent on the presence of a functional csrB gene (FIG. 5). Sixth, comparisons of sdiA wild type, mutant, and overexpressing strains confirmed that SdiA activates expression of a uvrY‘-’lacZ translational fusion (FIG. 7). In addition, sdiA was found to activate csrB expression through its effect on uvrY.

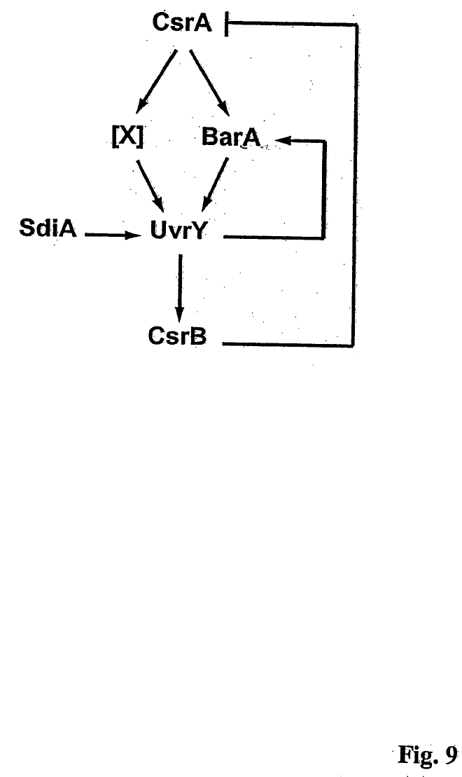

While the invention is not limited to any particular mechanism, the model shown in FIG. 9 presents the results of the examples herein, within the regulatory circuitry of the Csr system. The RNA binding protein CsrA is the key regulator of the Csr system, and indirectly activates the transcription of its RNA antagonist, CsrB, ˜20-fold. Although CsrA binds to CsrB RNA, it does not alter its chemical decay rate, which has a half-life of ˜2 min. The effects of CsrA on csrB expression appear to be completely dependent upon UvrY (FIG. 4), which is a direct activator of csrB expression (FIG. 6). While barA is involved in the circuitry, mutagenesis (FIGS. 1 and 2) and complementation studies (FIG. 4) suggest that CsrA can activate csrB expression independently of BarA. BarA has been shown to phosphorylate UvrY, and activation of UvrY by BarA likely occurs by phosphorylation. In addition, CsrA may cause a second kinase to phosphorylate UvrY. The relative effects of CsrA, UvrY and BarA on csrB expression and complementation studies with these genes collectively suggest that a BarA-independent mechanism also activates UvrY. The model in FIG. 9 also depicts the finding that UvrY stimulates barA expression, indicative of a positive autoregulatory loop within this system. Finally, SdiA activates uvrY expression, and in this way affects csrB expression. Although the sigma factor RpoS or sigma^{-s} is important in stationary phase regulation, and rpoS transcription appears to be activated by barA, neither CsrA protein n or CsrB RNA levels were significantly affected by rpoS disruption.

The UvrY/BarA two component signal transduction system was recently recognized in E. coli, and biochemical and genetic evidence was presented to demonstrate direct phosphotransfer from BarA to UvrY. BarA of E. coli appears to be involved in the bacterial adaptive responses against hydrogen peroxide-mediated stress by activating transcription of the rpoS gene, which encodes a sigma factor involved in the expression of stationary phase and stress response genes.

A sequence which overlaps the −35 region of the uvrY promoter is a SdiA box that is important to activation of uvrY transcription. E. coli, Salmonella and Klebsiella spp. are not known to synthesize the AHL and have no apparent AHL synthase genes. Nevertheless, SdiA may provide a means of recognizing other species (e.g. within the intestinal tract) and modulating csrB expression through effects on uvrY. However, in studies of the Salmonella sdiA gene, the most active AHL derivative had no effect on the expression of the disclosed csrB-lacZ fusion in E. coli.

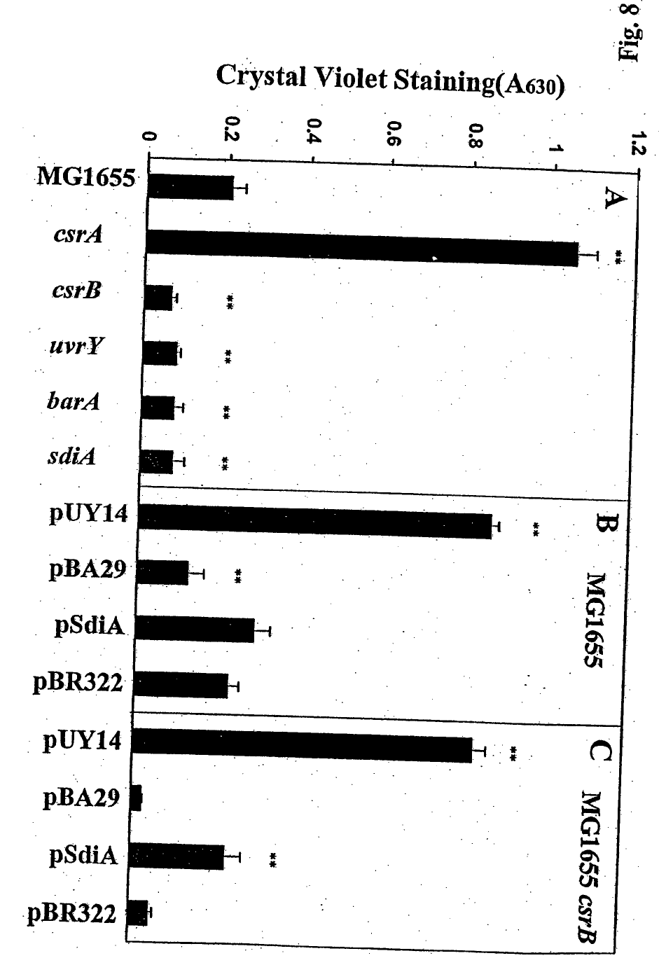

CsrA is a repressor of biofilm formation, while CsrB activates biofilm formation. The gratuitous induction of csrA in a preformed biofilm caused it to disperse by liberating viable planktonic cells. The effect of CsrA on biofilm formation was mediated largely through its role as a regulator of intracellular glycogen synthesis and turnover in the stationary phase of growth. The most striking finding noted upon examination of biofilm formation by strains in the present study was that ectopic expression of uvrY caused several-fold increase in biofilm formation, which was almost as great as the increase caused by a csrA mutation (FIG. 8). UvrY was able to activate biofilm formation independently of CsrB. The gacA gene of Pseudomonas aeruginosa, which is homologous to uvrY, appears to activate biofilm formation. A number of possible factors that are required for biofilm formation were examined, including twitching and swarming motililty, alginate biosynthesis, and autoinducer production, but none accounted for the regulatory effect of GacA.

In an embodiment of the invention there is provided a method of reducing biofilm formation by increasing CsrA levels in a bacterial strain. In some instances, the bacterial strain is an E. coli strain, a Salmonella strain, a Klebsiella strain or a related gamma proteobacteria. CsrA activity may be increased by decreasing transcription of csrB, by increasing transcription of csrA DNA, by reducing translation of csrA mRNA, or by reducing the half-life of csrA RNA and/or CsrA.

Agents causing increased CsrA activity are referred to herein as “CsrA stimulators”, and include inhibitors of csrB transcription, and stimulators of CsrA transcription and/or translation.

In an embodiment of the invention there is provided a method of increasing biofilm formation by decreasing CsrA levels in a bacterial cell. In some instances, the bacterial cell will be an E. coli cell, a Salmonella cell, a Klebsiella cell or a related gamma proteobacteria. CsrA levels may be decreased by increasing the level of csrB transcripts in the bacterial cell.

As used herein, the level of RNA or protein is “increased” if it is at least 10% higher than is found in normal cells of the same type under the same growth conditions.

In an embodiment of the invention there is provided a method of increasing CsrB levels in a bacterial cell by increasing the levels of active UvrY in the cell. Levels of active UvrY in the cell may be increased by increasing the expression of one or both of BarA and/or SdiA, by increasing uvrY transcription or uvrY RNA translation, by increasing the rate of UvrY phosphorylation, by decreasing the rate of UvrY dephosphorylation, or by increasing the half-life of uvrY mRNA and/or UvrY in the cell.

In an embodiment of the invention there is provided a method of modulating biofilm formation in a bacterial cell, comprising modulating the level of active UvrY in the cell. In some instances, the bacterial cell is an E. coli cell. In some instances, the cell is a Salmonella cell or a related gamma proteobacteria.

In an embodiment of the invention there is provided a modulator of CsrA activity in a bacterial cell, said modulator comprising a nucleotide sequence containing the element CAGGAUG. The element may be repeated preferably from about 2 to about 100 times. In some instances, repeats of between 5 and 50 elements will be desirable. In some instances, repeats of 18 elements will be desirable. In some instances, repeats of 19 elements will be required.

In an embodiment of the invention there is provided a method of increasing biofilm formation in a bacterial cell, comprising inducing in that cell the expression of a repeated nucleotide sequence, said sequence being CAGGAUG (SEQ ID NO: 1).

In an embodiment of the invention there is provided a method of reducing biofilm formation, comprising increasing BarA levels in a bacterial cell. In some instances, the bacterial cell is preferably an E. coli cell, Salmonella cell, or a related gamma proteobacteria.

In an embodiment of the invention there is provided a method of decreasing biofilm formation comprising modulating UvrY phosphorylation.

In an embodiment of the invention there is provided a method of modulating biofilm formation in a bacterial cell, comprising modulating the level of phosphorylation of UvrY in the cell. In some instances, the cell is preferably and E. coli or Salmonella cell or a related gamma proteobacteria.

In an embodiment of the invention there is provided a modulator of biofilm formation in a bacterial cell, said modulator comprising an amino acid sequence selected from a portion of the amino acid sequence of CsrA shown to selectively bind RNA of biofilm-related genes under stringent conditions in vitro.

In an embodiment of the invention there is provided a method of modulating the expression of an RNA containing at least one of the nucleotide sequences UGCACACRRNYYGUGUGUG (SEQ ID NO: 2), UGCACACYYNRRGUGUGUG (SEQ ID NO: 3), UGCACACGGAUUGUGUGUG (SEQ ID NO: 4), and RNA sequences at least 90% homologous to at least one of these sequences, wherein Y represents a pyrimidine, R represents a purine and N represents either a purine or a pyrimidine, comprising providing an RNA binding agent specifically recognizing the sequence.

In some instances the RNA binding agent is a peptide or protein selected from a portion of the amino acid sequence of CsrA shown to selectively bind RNA of biofilm-related genes under stringent conditions in vitro, or an amino acid sequence at least 80% homologous thereto which specifically binds the same nucleotide sequence with at least 50% of the affinity of the amino acid sequence. In some instances the RNA binding agent is a small molecule having a binding surface sharing substantially the same hydrophobicity, charge distribution, hydrogen-bonding ability, Van der Waal's size and shape as the portion of the amino acid sequence which binds the nucleotide sequence, and which specifically binds the nucleotide sequence with at least 50% of the affinity of the amino acid sequence.

In an embodiment of the invention there is provided a method of identifying modulators of biofilm formation. This method comprises identifying agents which modulate the level of CsrA in a bacterial cell, such as an E. coli cell, Salmonella cell, or a related gamma proteobacteria. In one embodiment of this method, there is provided a reporter gene system approach to identifying inhibitors, comprising a fused nucleotide containing genetic material encoding a reporter (such as lacZ) fused to the regulatory region of the biofilm formation modulating gene of interest (such as uvrY, csrB, csrA, sdiA, and/or barA). In another embodiment of the method there is provided a mobility shift approach to identifying inhibitors of CsrA, comprising identifying agents which slow the migration of purified CsrA through a matrix which retards mobility based on molecular weight or size. In another embodiment of the method there is provided a transcriptional activation assay useful in identifying inhibitors of compounds upstream of csrB, comprising assaying in vitro activation of csrB expression by assaying csrB RNA levels.

Inhibitors of particular interest include kinase inhibitors, as well as small molecules having surfaces available for interaction with a similar Van der Waals radius, charge, hydrogen bonding affinity and shape as known interaction domains of UvrY, CsrB, SdiA or CsrA.

In an embodiment of the invention there is provided an inhibitor of CsrA activity, comprising a nucleotide containing two or more repeats of the sequence element CAGGAUG.

In an embodiment of the invention there is provided a stimulator of biofilm formation, comprising a nucleotide containing two or more repeats of the sequence element CAGGAUG.

In an embodiment of the invention there is provided a modulator of biofilm formation, comprising an amino acid selected from a portion of the amino acid sequence of CsrA shown to selectively bind RNA of biofilm-related genes under stringent conditions in vitro

In an embodiment of the invention there is provided a modulator of biofilm formation, comprising a small molecule having a binding surface sharing the shape, hydrophobicity, hydrogen bonding affinity and charge distribution of the CsrA region which binds to flhDC transcripts.

In an embodiment of the invention there is provided a modulator of biofilm formation, comprising a small molecule having a binding surface sharing the shape, hydrophobicity, hydrogen bonding affinity and charge distribution of the CsrA region which binds to glgCAP transcripts.

In an embodiment of the invention there is provided a method of modulating glycogen biosynthesis and catabolism comprising modulating the level of CsrA in a bacterial cell. In some instances, the cell is preferably and E. coli or Salmonella cell, or a related gamma proteobacteria.

In an embodiment of the invention there is provided a method of modulating glycogen biosynthesis, comprising inducing the presence within a bacterial cell of a nucleotide having two or more repeats of the sequence CAGGAUG.

In an embodiment of the invention there is provided a method of modulating glycogen biosynthesis in a bacterial cell comprising administering to the cell a compound having an RNA binding region sharing substantially the same shape, size, charge distribution, and hydrogen bonding affinity as an amino acid sequence selected from a portion of the amino acid sequence of CsrA shown to selectively bind RNA of biofilm-related genes under stringent conditions in vitro in situ and which specifically binds RNA sequence UGCACACRRNYYGUGUGUG, UGCACACYYNRRGUGUGUG, UGCACACGGAUUGUGUGUG, or RNA sequences at least 90% homologous to at least one of these sequences, wherein Y represents a pyrimidine, R represents a purine and N represents either a purine or a pyrimidine, under physiological conditions with at least 50% of the affinity of CsrA for this RNA sequence.

In an embodiment of the invention there is provided a method of improving recovery of a mammalian patient suffering from infection by bacteria forming biofilm, comprising administering to the patient in a form suitable for uptake by the bacteria, an inhibitor of biofilm formation. In some instances, the bacteria is preferably an E. coli or Salmonella bacteria, or a related gamma proteobacteria. In some instances, the patient is a human or domestic animal.

In an embodiment of the invention there is provided a method of reducing bacterial load in a mammalian patient suffering from infection by bacterial forming biofilm, comprising administering to the patient, in a form suitable for uptake by the bacteria, a compound having an RNA binding region sharing substantially the same shape, size, charge distribution, and hydrogen bonding affinity as a sequence selected from a portion of the amino acid sequence of CsrA shown to selectively bind RNA of biofilm-related genes under stringent conditions in vitro in situ in CsrA, and which specifically binds UGCACACRRNYYGUGUGUG, UGCACACYYNRRGUGUGUG, UGCACACGGAUUGUGUGUG, or RNA sequences at least 90% homologous to at least one of these sequences, wherein Y represents a pyrimidine, R represents a purine and N represents either a purine or a pyrimidine, with at least 50% of the affinity of CsrA for this sequence.

Agents may be placed into a form suitable for uptake by bacteria by encapsulation using membranes selected to encourage uptake by bacteria, or other known means.

In an embodiment of the invention there is provided a modulator of biofilm formation by a bacterial cell comprising an agent having an AHL binding domain and a DNA binding domain and having at least 50% of the activity of SdiA in stimulating UvrA under physiological conditions.

In some instances the AHL binding domain is an amino acid sequence between 80% and 99% homologous to the SdiA DNA binding domain. In some instances the DNA binding domain is an amino acid sequence between 80% and 99% homologous to the SdiA DNA binding domain.

### EXAMPLES

Materials and Methods

Strains, plasmids and phage. The bacterial strains, plasmids, and bacteriophage used in this study are listed in Table 1.

Media and growth conditions. Luria-Bertani medium (34) was used for routine cultures. Kornberg medium (1.1% K₂HPO₄, 0.85% KH₂PO₄, 0.6% yeast extract containing 0.5% glucose for liquid medium) was used to grow cultures for the gene expression assays and Northern blot analysis. M63 medium supplemented with glucose (0.4%), thiamine (5 μg/ml), adenine and thymine (50 μg/ml), calcium pantothenate (1 μg/ml), and serine, glycine, methionine (100 μg/ml each), was used for the selection of rel⁺ barA mutants (55). Tryptone broth (pH 7.4) contained 1% tryptone and 0.5% NaCl. Colonization factor antigen (CFA) medium (pH 7.4) contained 1% casamino acids, 0.15% yeast extract, 0.005% MgSO₄, and 0.0005% MnCl₂ (16). The following antibiotics were added, as required, at the following concentrations: chloramphenicol, 20 μg/ml; kanamycin, 50 μg/ml; ampicillin, 100 μg/ml; and tetracycline, 10 μg/ml, except that ampicillin and kanamycin were used at 50 and 40 μg/ml, respectively, during the construction of the uvrY‘-’lacZ fusion, and kanamycin was used at 100 μg/ml for the selection of csrA::kanR strains. All cultures that were used for gene expression assays were grown at 37° C. with rapid rotary shaking (48).

Molecular and genetic techniques. P1vir transduction or cotransduction of resistance markers, subcloning, PCR amplification, and molecular genetic techniques were performed by standard procedures (34, 50).

The plasmid pBarA was constructed by PCR amplification of the barA gene from −270 to the end of the barA coding region from E. coli MC4100 chromosomal DNA using Pfu polymerase and the primers 5′-GAGAATGCATACGCCAAAATGAGGACAG (SEQ ID NO: 5) and 5′-GCGGATCCACTCGACMGACATCCATTA (SEQ ID NO: 6). The resultant product was cloned directly into the pGEM-T vector (Promega) using the T-overhang, with the barA gene position in a clockwise direction. A 0.5-kb BamHI-EcoRI fragment containing csrA from pCSR10 (48), a 1.3 kb EcoRI-HindIII fragment containing uvrY from pCA9505 (36), and a 3.0-kb Ncol-Notl fragment containing barA from pBarA were treated with the Klenow fragment of DNA polymerase I and were individually subcloned into the blunt-ended Vspl site of pBR322 to generate p CRA16, p UY14, and pBA29, respectively. The open reading frames of the above genes are oriented in the same direction as bla in the vector.

Special precautions were required for two of the P1 vir transductions of this study, as follows. The barA::kanR mutation was transduced from AKP014 (42) into MG1655 with selection for kanamycin resistance. Because AKP014 is a relA mutant, and relA is separated by only 1.4 Kb of DNA from barA, a relA wild type transductant was selected by virtue of its ability to grow on M63 supplemented medium, and was designated as BA MG1655. The uvrY::cam mutation was transduced from AKP023 (42) into CF7789. Because AKP023 is a flhD mutant, and flhD is located 21 Kb away from uvrY, a motile transductant was identified using the plate assay for motility, and designated as UY CF7789. The other barA or uvrY mutants were generated by P1vir transduction from BA MG1655 or UY CF7789, respectively.

Construction of a chromosomal uvrY‘-’lacZ translational fusion. A 572-bp fragment containing the upstream regulatory region and 12 codons of uvrY was amplified from MG1655 DNA by PCR using the primers 5′-CAGCATCGCTTTCAGGCAGGAGACTTC (SEQ ID NO: 7) and 5′-CAGTTCGTGGTCATCMCMGTAGMCG (SEQ ID NO: 8) and was treated with T4 DNA polymerase and polynucleotide kinase and subcloned into the Smal site of pMLB1034 (51). The resulting plasmid, pUZ9, contained 26 codons of yecF, which is upstream from and divergently oriented with respect to uvrY (7), the complete upstream flanking region of uvrY, 12 codons of the uvrY coding region, and an in frame uvrY‘-’lacZ translational fusion. DNA sequencing was performed to confirm the presence of the correct fusion and the absence of PCR-generated mutations. The uvrY‘-’lacZ fusion in pUZ9 was moved into the E. coli CF7789 chromosome using the lambda-InCh1 system as described (9). The resulting strain, KSY009, which was chosen for subsequent studies was Amp^{r} Kan^{s} and was no longer temperature sensitive. The presence of the uvrY‘-’lacZ translational fusion was confirmed by PCR analysis, as recommended (9). Oligonucleotide primers used in this study were synthesized by Integrated DNA Technologies Inc., Coralville, Iowa.

Motility assay. The plate assay was initiated by stabbing a colony from an overnight culture into semi-solid agar (tryptone broth or CFA medium solidified with 0.35% agar). The plates were kept in a humidified incubator at 30° C. and examined at −16 h of growth (59).

β-Galactosidase and total protein assays. β-Galactosidase activity was assayed in 10-min reactions, as described previously (46). Total protein was measured by the bicinchoninic acid method using bovine serum albumin as the protein standard (53).

Purification of His6-tagged UvrY. His6-tagged UvrY was purified as described previously, except that purified protein was dialyzed against 10 mM Tris-OAc (pH 8.0) containing 25% glycerol and concentrated in Centricon 10 units (Amicon) (42).

In vitro transcription-translation. Effects of UvrY protein on csrB-lacZ expression were examined using S-30 extracts prepared from a uvrY mutant strain (UYCF7789), as previously described (31, 48), except that reaction volumes were scaled down to 28 pl. Radiolabeled proteins were detected by fluorography using sodium salicylate (10) and methionine incorporation into the LacZ polypeptide was quantified by liquid scintillation counting of H₂O₂-solubilized gel sections (48).

Quantitative biofilm assay. Overnight cultures were inoculated 1:100 into fresh medium. In the microtitre plate assay, inoculated cultures were grown in a 96-well polystyrene microtitre plate. Growth of planktonic cells was determined by absorbance at 600 nm or total protein assay. Biofilm was measured by discarding the medium, rinsing the wells with water (three times), and staining bound cells with crystal violet (BBL) (O'Toole, Mol. Microbiol. 30: 295 (1998)). The dye was solubilized with 33% acetic acid (EM Science, Gibbstown, N.J.), and absorbance at 630 nm was determined using a microtitre plate reader (DynaTech, Chantilly, Va.). For each experiment, background staining was corrected by subtracting the crystal violet bound to uninoculated controls. All comparative analyses were conducted by incubating strains within the same microtitre plate to minimize variability. To confirm that observed effects on biofilm formation in microtitre wells were not surface specific, cultures were grown and tested simultaneously in new borosilicate glass test tubes (18 mm).

Northern hybridization. RNA isolation, riboprobe synthesis and Northern blotting were conducted. Total cellular RNA (5 μg) was separated on formaldehyde agarose (1%) gels, transferred overnight onto positively charged nylon membranes (Boehringer Mannheim) in 20×SSC (1×SSC is 0.15 M NaCl plus 0.015 M sodium citrate), and immobilized by baking at 120° C. for 30 min (36). Prehybridization, hybridization to DIG-labelled riboprobes (2 μl of probe per 10 ml of prehybridization buffer), and membrane washing were conducted using the DIG Luminescent Detection kit for nucleotide acids (Boehringer Mannheim), according to the manufacturer's instructions. The resulting chemiluminescent signals were detected using Kodax X-Omat-AR film and were quantified by phosphorimaging using a GS-525 phosphorimager (Bio-Rad, Hercules, Calif.) with a chemiluminescent screen. Isolated CsrB RNA (23) was used to generate a standard curve for CsrB signal quantitation. Cellular CsrB transcript levels were quantified within the linear response range of the purified standard. Phosphorimaging data were analyzed using Molecular Analyst version 2.1.2) software and Microsoft Excel. For RNA decay studies, strains were grown to the transition to stationary phase (A₆₀₀ of 5.1 and 6.5 for the csrA wild type and mutant, respectively), rifampin (200 μg/ml [final concentration]) was added, and samples were collected at 0, 2, 4, 6, 8, and 12 min following rifampin addition. Samples (1.5 ml) were immediately centrifuged for 15 s at 15,000 × g, the spent medium was discarded, and the cells were frozen on dry ice-ethanol and stored at −80° C. pending RNA isolation. RNA (5 μg) was separated on formaldehyde agarose (1.2%) gels, blotted by capillary transfer onto positively charged nylon membranes (Boehringer Mannheim), and immobilized by baking at 120° C. for 30 min. A digoxygenin (DIG)-labelled csrB riboprobe was synthesized from pSPT18-CsrB using the DIG Luminescent Detection kit for nucleic acids, as described by the manufacturer (Boehringer Mannheim). The blot was pre-hybridized and hybridized at a probe concentration of 50 ng/ml using Perfecthyb Plus™ hybridization buffer (Sigma Chemical). Signal detection used the commercial protocol (Boehringer Mannheim), except that incubation in blocking solution was extended for an additional 10 h. Chemiluminescent signals were detected using Kodak X-Omat-AR film. In addition, signals were quantified by phosphorimaging using a GS-525 Phosphorimager (Bio-Rad). Phosphorimaging data were analyzed using Molecular Analyst (version 2.1.2) software (Bio-Rad). Prior to blotting, the gels were stained with ethidium bromide and rRNA bands were photographed. The resulting signals were quantified by densitometry, and values used to correct for minor variations in sample loading.

### Example 1

### Effects of uvrY and barA on the in vivo Expression of csrB

Northern hybridization was used to determine if UvrY or BarA affect CsrB RNA levels (FIG. 1). RNA was isolated from MG1655 and its isogenic uvrY and barA mutants at ˜2 h post-exponential phase, which is optimal for CsrB accumulation. CsrB RNA levels were decreased ˜60% in the barA mutant, and ˜98% in the uvrY mutant, relative to the parent strain. These results indicated that UvrY is an important regulator of csrB expression and that BarA influences csrB expression to a lesser extent.

To further determine if uvrY and barA regulate expression of csrA and csrB, expression from chromosomal csrA‘-’lacZ translational or csrB-lacZ transcriptional fusions was examined in wild type, uvrY or barA mutant strains. The specific β-galactosidase activity from the csrA‘-’lacZ fusion was not altered by either mutation (FIG. 2A), while the activity from the csrB-lacZ fusion was dependent upon both uvrY and barA (FIG. 2B). The uvrY mutation reduced csrB-lacZ expression ˜95% or 20-fold, whereas, the barA mutant exhibited a decrease of ˜70%. BarA is a member of the subclass of tripartite sensor kinases and UvrY is the cognate response regulator for BarA. These results indicate that BarA-phosphorylated UvrY activates transcription of the csrB gene, and that either unphosphorylated UvrY can activate csrB expression to a lesser extent or that UvrY is activated by an alternative phosphoryl donor.

### Example 2

### Effects of csrA and uvrY on barA Expression

To further examine the regulatory interactions of the CsrA/CsrB and BarA/UvrY systems, the effects of CsrA and UvrY on barA expression were examined by monitoring expression of a chromosomal barA-lacZ transcriptional fusion in wild type, csrA or uvrY mutant strains. The wild type strains exhibited ˜2-fold greater β-galactosidase activity than their isogenic csrA (FIG. 3A) or uvrY mutants (FIG. 3B). Because HS703 was kanamycin resistant, this csrA isogenic strain pair was constructed by cotransduction of the csrA::kanR mutation along with a closely-linked tetR marker from TR1-5 CAG18642 into HS703 (barA-lacZ). Tet^{r} transductants were screened for the csrA glycogen phenotype to distinguish csrA wild type and mutant colonies, and isogenic csrA mutant and wild type strains, which each contained the tetR marker that was used for cotransduction, were compared in the assays (FIG. 3A). The tetR mutation itself increased barA-lacZ expression ˜2-fold (compare the isogenic parent strains, CAGHS703 and HS703 in FIG. 3A and B). The gene disrupted by t he tetR marker is srID (gutD) and encodes the glucitol-6-phosphate dehydrogenase of the glucitol operon. The basis of its effect on barA expression is unknown. In conclusion, UvrY and CsrA each exhibit modest stimulation of barA expression.

### Example 3

### Effects of csrA, barA, and uvrY on uvrY Expression

Expression from a uvrY‘-’lacZ translational fusion in csrA wild-type and mutant strains was examined. Expression from this gene fusion was not affected by a csrA mutation. Overexpression of csrA from a plasmid resulted in only slight activation of this fusion, which is not likely to be biologically relevant. Similarly, the disruption of barA or uvrY had no effect on the expression of this uvrY‘-’lacZ fusion.

### Example 4

### Complementation Studies: Effects of Ectopic Expression of csrA, uvrY or barA on csrB-lacZ Expression in csrA, uvrY, and barA Mutants

CsrA is a strong activator of csrB transcription. This effect is indirect. uvrY, and barA activate csrB expression (FIGS. 1 and 2), and csrA modestly stimulates barA expression (FIG. 3). Complementation studies were conducted to further delineate the regulatory circuitry of this system. Multicopy plasmids containing either csrA or uvrY restored csrB-lacZ expression in a csrA mutant background (FIG. 4). Only uvrY could restore csrB-lacZ expression in a uvrY mutant (FIG. 4). barA, uvrY or csrA were able to enhance csrB-lacZ expression in a barA strain background (FIG. 4). These results suggest a late or terminal role for UvrY in a signalling pathway to csrB. These results indicate that csrA influences csrB expression through BarA-dependent and independent mechanisms. In addition, csrA has no effect on csrB expression in the uvrY mutant background, indicating that its role in csrB expression is completely dependent upon UvrY. While the invention is not limited to any particular mechanism, this suggests that CsrA is involved in both BarA-dependent and -independent pathways for UvrY activation. Taken in context, the failure of barA to complement a csrA defect also suggests that CsrA may affect BarA function, e.g. CsrA may indirectly influence BarA activation.

### Example 5

### Effects of uvrY and barA on CsrB-activated Genes

Expression of a chromosomal glgCA‘-’lacZ translational fusion and a plasmid-encoded glgC‘-’lacZ translational fusion were monitored. The effect of uvrY disruption was similar to that of csrB, and resulted in a modest decrease in glgCA‘-’lacZ expression (FIG. 5A). Likewise, overexpression of uvrY yielded the opposite effect of uvrY and csrB disruption (FIG. 5B). Ectopic expression of uvrY from the multicopy plasmid pUY14 in a csrB null mutant strain resulted in modest to negligible effects on glgCA‘-’lacz (FIG. 5C), suggesting that UvrY affects expression of glgCAP primarily through its role as an activator of csrB. The uvrY and barA mutations also decreased glgC‘-’lacZ expression from pCZ3-3 ˜2-fold.

### Example 6

### UvrY Activates csrB Expression in Vitro

In vitro transcription-translation of pCBZ1-encoded csrB-lacZ transcriptional fusion was examined in S-30 extracts prepared from the uvrY mutant, UY CF7789, in the presence of various concentrations of purified UvrY protein. The expression of the pCBZ1-encoded csrB-lacZ fusion was activated ˜20-fold by uvrY in vivo, as determined in uvrY wild type versus mutant strains. As shown in FIG. 6, in vitro synthesis of the LacZ protein was stimulated ˜6-fold in the presence of 2.3 μM UvrY protein subunits, a concentration that saturated the reaction. Synthesis of the LacZ polypeptide was also detected in reactions with the control vector, pGE593. This was likely due to read-through transcription. However, in contrast to the reactions with pCBZ1, LacZ expression from the control vector was not stimulated by UvrY. Since pCBZ1 contained the upstream region of csrB (−242 to +4), this indicates that UvrY activates csrB transcription, presumably by binding to csrB DNA. In this experiment, recombinant UvrY protein was used as isolated from the cell. Since BarA is required for maximal expression of csrB in vivo (FIGS. 1 and 2), it is likely that phosphorylated UvrY activates csrB transcription. UvrY protein may be phosphorylated prior to or during the S-30 reaction, or that unphosphorylated UvrY may bind to DNA and activate transcription, albeit at reduced affinity relative to the phosphorylated form.

### Example 7

### Effects of sdiA on Expression of csrA, csrB, and uvrY

SdiA is a LuxR homologue that possesses a putative AHL binding domain and a second domain for binding DNA. Genomic array studies indicated that an increase in the copy number of sdiA significantly increases the levels of uvrY mRNA. Expression from chromosomal csrA‘-’lacZ, csrB-lacZ, and uvrY‘-’lacZ fusions in isogenic sdiA mutant and wild type strains was examined. Expression from these fusions in strains containing a plasmid clone of sdiA, pSdiA, or the cloning vector, pBR322 was also compared. No significant effect of SdiA was observed for csrA‘-’lacZ expression (FIGS. 7A, B). However, expressions from csrB-lacZ and uvrY‘-’lacZ fusions were partially dependent upon sdiA, as they were decreased by the sdiA mutation (FIGS. 7C, E) and increased 1.5- fold and ˜6-fold, respectively (FIGS. 7D, F), by sdiA overexpression. In order to determine whether sdiA regulates csrB expression via its effect on uvrY, a complementation test was conducted. Expression from the csrB-lacZ fusion in the uvrY mutant was no longer affected by sdiA overexpression, suggesting that sdiA stimulates csrB expression through its effects on uvrY. No effect of AHL, over a broad concentration range, on lacZ fusions for csrA, csrB or uvrY, in sdiA wild type or mutant strains was observed.

### Example 8

### Effects of uvrY, barA, or sdiA on Biofilm Formation

CsrA represses biofilm formation, while CsrB activates this process. The effects of uvry, barA, and sdiA on biofilm formation were monitored in static cultures using the microtiter plate assay, which measures the binding of crystal violet to adherent cells of the biofilm. The ectopic expression of uvrY activated biofilm formation several-fold (FIG. 8B). This effect was almost as great as that of a csrA knockout mutation. In addition, uvrY overexpression activated biofilm formation in a csrB mutant strain background, indicating that UvrY has effects on biofilm formation that are mediated independently of csrB. More modest effects were observed for knockouts of csrB, uvrY, barA and sdiA, which were still statistically significant (FIG. 8A). The parent strain (MG1655) formed ˜3-fold more biofilm than each of these mutants. Overexpression of barA caused a modest decrease in biofilm formation. The increased gene dosage of sdiA caused a modest increase in biofilm formation.

Thus, there has been provided compounds and methods for modulating bacterial functions.

TABLE 1
Bacterial strains, plasmids, and phages

| Strain, plasmid, | |
|---|---|
| or phage | Description |
| Strains | |
| AKP014 | MC4100 barA::kanR |
| AKP023 | MC4100 uvrY::cam |
| BA MG1655^{a} | MG1655 barA::kanR (from AKP014) |
| CAG18642 | zfh-3131::Tn10; 57.5 min, (near csrA) |
| CF7789 | MG1655 ΔlaclZ (Mlul) |
| DHB6521 | SM551 λlnCh1 (Kan^{r}) |
| HS703 | MC4100 barA::λplacMu53 [φ(barA-lacZ)1010] |
| KSA712 | CF7789 Δ(att-lom)::bla φ(csrA‘-’lacZ)1(Hyb) Amp^{r} |
| | Kan^{s} |
| KSB837 | CF7789 Δ(att-lom)::bla φ(csrB-lacZ)1(Hyb) Amp^{r} |
| | Kan^{s} |
| KSY009 | CF7789 Δ(att-lom)::bla φ(uvrY‘-’lacZ)1(Hyb) Amp^{r} |
| | Kan^{s} |
| KSGA18 | CF7789 φ(glgA::lacZ) (λplacMu15) |
| MG1655 | Prototrophic |
| RG1-B MG1655^{a} | csrB::cam |
| SM551(=DHB6501) | F⁻ λ⁻ λ^{s} Δlac(MS265) mel NalA^{r}supF58 (=sulll⁺) |
| TR1-5 MG1655^{a} | csrA::kanR |
| UY CF7789^{a} | CF7789 uvrY::cam (from AKP023) |
| WX2 | Δlac-pro met pro zzz::Tn10 thy supD r_{K}⁻ m_{K}⁻ |
| | sdiA::kanR |
| Plasmids | |
| pCBZ1 | pGE593 φ(csrB-lacZ) |
| pCZ3-3 | pMLB1034 φglgC‘-’lacZ) |
| pUZ9 | pMLB1034 φ (uvrY‘-’lacZ) |
| pCSR10 | Minimal csrA in pUC19 |
| pCRA16 | csrA in blunt-ended Vspl site of pBR322, Tet^{r} |
| pCA9505 | Carries uvrY gene, Amp^{r} |
| pUY14 | uvrY in blunt-ended Vspl site of pBR322, Tet^{r} |
| pBarA | barA in pGEM-T, Amp^{r} |
| pBA29 | barA in blunt-ended Vspl site of pBR322, Tet^{r} |
| pSdiA | sdiA in EcoRl site of pBR322, Amp^{r} Tet^{r} |
| pSPT18-CsrB | Plasmid for the csrB riboprobe synthesis |
| pGE593 | Vector for lacZ transcriptional fusions; Amp^{r} |
| pMLB1034 | Vector for lacZ translational fusions; Amp^{r} |
| pGEM-T | T-cloning vector, Amp^{r} |
| pBR322 | Cloning vector; Amp^{r} Tet^{r} |
| Bacteriophages | |
| P1vir | Strictly lytic P1 |
| λInCh1 | For genomic insertions; Kan^{r} |

^{a}Strain designations containing the prefix TR or TR1-5, RG or RG1-B, UY, BA, or SA indicate that the mutant allele csrA::kanR, csrB::cam, uvrY::cam, barA::kanR, or sdiA::kanR, respectively, was introduced by P1vir transduction.

References

1. Blattner, F. R., G. Plunkett, C. A. Bloch, N. T. Perna, V. Burland, M. Riley, J. Collado-Vides, J. D. Glasner, C. K. Rode, G. F. Mayhew, J. Gregor, N. W. Davis, H. A. Kirkpatrick, M. A. Goeden, D. J. Rose, B. Mau, and Y. Shao. 1997. The complete genome sequence of Escherichia coli K-1 2. Science. 277:1453-1474.

2. Boyd, D., S. D. Weiss, J. C. Chen, and J. Beckwith. 2000. Towards single copy gene expression systems making gene cloning physiologically relevant: Lambda InCh, a simple Escherichia coli plasmid-chromosome shuttle system. J. Bacteriol. 182:842-847.

3. Chamberlain, J. P. 1979. Fluorographic detection of radioactivity in polyacrylamide gels with the water-soluble flour, sodium salicylate. Anal. Biochem. 98:132-135.

4. Evans, D. G., D. J. Evans, Jr, and W. Tjoa. 1997. Hemagglutination of human group A erythrocytes by enterotoxigenic Escherichia coli isolated from adults with diarrhea: correlation with colonization factor. Infect. Immun. 18:330-337. teriol. 179:4639-4642.

5. Liu, M. Y., G. Gui, B. Wei, J. F. Preston III, L. O akford, U. Yuksel, D. P. Giedroc, and T. Romeo. 1997. The RNA molecule CsrB binds to the global regulatory protein CsrA and antagonizes its activity in Escherichia coli J. Biol. Chem. 272:17502-17510.

6. Miller, J. H. 1972. Experiments in molecular genetics. Cold Spring Harbor Laboratory Press, Cold Spring Harbor, N.Y.

7. Moolenaar, G. F., C. A. van Sluis, C. Backendorf, and P. van de Putte. 1987. Regulation for the Escherichia coli excision repair gene uvrC. Overlap between the uvrC structural gene and the region coding for a 24 kD protein. Nucleic Acids Res. 15:4273-4289.

8. Pernestig, A.-K., Ö. Melefors, and D. Georgellis. 2001. Identification of UvrY as the cognate response regulator for the BarA sensor kinase in Escherichia coli. J. Biol. Chem. 276:225-231.

9. Romeo, T., J. Black, and J. Preiss. 1990. Genetic regulation of glycogen biosynthesis in Escherichia coli: in vivo effects of the catabolite repression and stringent response systems in glg gene expression. Curr. Microbiol. 21:131-137.

10. Romeo, T., and J. Preiss. 1989. Genetic regulation of glycogen biosynthesis in Escherichia coli: in vitro effects of cyclic AMP and guanosine 5′-diphosphate 3′-diphosphate and analysis of in vivo transcripts. J. Bacteriol. 171:2773-2782.

11. Sambrook, J., E. F. Fritsch, and T. Maniatis. 1989. Molecular cloning: a laboratory manual, 2nd ed. Cold Spring Harbor Laboratory Press, Cold Spring Harbor, N.Y.

12. Silhavy, T. J., M. L. Berman, and L. W. Enquist. 1984. Experiments with gene fusions. Cold Spring Harbor Laboratory, Cold Spring Habor, N. Y.

13. Smith, P. K., R. H. Krohn, G. T. Hermanson, A. K. Mallie, F. H. Gartner, J. D. Provensano, E. K. Fujimoto, N. M. Goeke, B. J. Olson, and D. C. Klenk. 1985. Measurement of protein using bicinchoninic acid. Anal. Biochem. 150:76-85.

14. Uzan, M., and A. Danchin. 1976. A rapid test for the relA mutation in E. coli. Biochem. Biophys. Res. Commun. 69:751-758.

15. Wei, B. L., A.-M. Brun-Zinkernagel, J. W. Simecka, B. M. Prü, P. Babitzke, and T. Romeo. 2001. Positive regulation of motility and flhDC expression by the RNA-binding protein CsrA of Escherichia coli. Mol. Microbiol. 40:245-256.

We claim:

1. A method of reducing biofilm formation by increasing CsrA levels in a biofilm-forming bacterial strain.

2. The method of claim 1 wherein the bacterial strain is an E. coli strain, a Salmonella strain, a Klebsiella strain, or a related gamma proteobacteria.

3. A method of increasing biofilm formation by decreasing CsrA levels in a biofilm-forming bacterial strain.

4. The method of claim 3 in which the bacterial strain is one of an E. coli strain, a -ella strain, a Klebsiella strain, or a related gamma proteobacteria.

5. A method of increasing CsrB levels in a bacterial strain by increasing the levels of active UvrY in the strain.

6. The method of claim 5 wherein the bacterial strain is a biofilm-forming strain.

7. The method of claim 5 wherein levels of active UvrY in the strain are increased by increasing the expression of at least one of BarA and SdiA, increasing uvrY translation, increasing uvrY RNA translation, increasing the rate of UvrY phosphorylation, decreasing the rate of UvrY dephosphorylation, or increasing the half-life of uvrY mRNA or UvrY in the strain.

8. A method of modulating biofilm formation in a biofilm-forming bacterial strain, comprising modulating the level of active UvrY in the strain.

9. The method of claim 8 wherein the bacterial strain is an E. coli strain, a Salmonella strain, a Klebsiella strain, or a related gamma proteobacteria.

10. The method of claim 9 wherein the bacterial strain is an E. coli strain.

11. A modulator of CsrA activity in a bacterial cell, said modulator comprising an isolated nucleotide sequence containing the sequence element CAGGAUG.

12. The modulator of claim 11 wherein the sequence element is repeated between 2 and 100 times within a region of RNA no more than 1500 nucleotides long.

13. The modulator of claim 12 wherein the sequence element is repeated between 5 and 50 times.

14. The modulator of claim 13 wherein the sequence element is repeated 18 times.

15. The modulator of claim 13 wherein the sequence element is repeated 19 times.

16. Use of a modulator of claim 11 to modulate biofilm formation in a biofilm-forming bacterial strain.

17. Use of claim 16 wherein the strain is an E. coli strain, a Salmonella strain, a Klebsiella strain, or a related gamma proteobacteria.

18. A method of increasing biofilm formation in a biofilm-forming bacterial strain, comprising inducing in that strain increased levels of a repeated nucleotide sequence, said sequence being CAGGAUG.

19. The method of claim 18 wherein the bacterial strain is an E. coli strain, a Salmonella strain, a Klebsiella strain, or a related gamma proteobacteria.

20. A method of increasing biofilm formation in a biofilm-forming bacterial strain, comprising increasing BarA levels or activity in the bacterial strain.

21. The method of claim 20 wherein the bacterial strain is an E. coli strain, Salmonella strain, or a related gamma proteobacteria.

22. A method of decreasing biofilm formation in a biofilm-forming bacterial strain, comprising modulating UvrY phosphorylation.

23. A method of modulating biofilm formation in a bacterial strain, comprising modulating the expression level of uvrY in the strain.

24. The method of claim 23 herein the strain is an E. coli strain, Salmonella strain, or a related gamma proteobacteria.

25. A modulator of biofilm formation in a biofilm-forming bacterial strain, said modulator comprising an amino acid sequence selected from a portion of the amino acid sequence of CsrA shown to selectively bind RNA of biofilm-related genes under stringent conditions in vitro.

26. A method of modulating the expression of a RNA containing at least one of the nucleotide sequences UGCACACRRNYYGUGUGUG, UGCACACYYNRRGUGUGUG, UGCACACGGAUUGUGUGUG, and RNA sequences at least 90% homologous to at least one of these sequences, wherein Y represents a pyrimidine, R represents a purine and N represents either a purine or a pyrimidine, comprising providing an RNA binding agent specifically recognizing the sequence.

27. The method of claim 26 wherein the RNA binding agent is a peptide or protein selected from a portion of the amino acid sequence of CsrA.

28. The method of claim 27 wherein the RNA binding agent is an amino acid sequence at least 80% homologous to a region of CsrA shown to selectively bind RNA of biofilm-related genes under stringent conditions in vitro, which specifically binds the same nucleotide sequence with at least 50% of the affinity of the amino acid sequence.

29. A CsrA binding agent comprising a RNA containing at least one of the nucleotide sequences UGCACACRRNYYGUGUGUG, UGCACACYYNRRGUGUGUG, UGCACACGGAUUGUGUGUG, or RNA sequences at least 90% homologous to at least one of these sequences, wherein Y represents a pyrimidine, R represents a purine and N represents either a purine or a pyrimidine and a physiologically acceptable carrier.

30. A method of identifying modulators of biofilm formation, comprising identifying agents which modulate the level of CsrA in a biofilm-forming bacterial strain.

31. The method of claim 30 wherein the bacterial strain is an E. coli strain, Salmonella strain, or a related gamma proteobacteria.

32. The method of claim 30 wherein agents are identified using a reporter gene system approach to identifying inhibitors, comprising a fused nucleotide containing genetic material encoding a reporter fused to the regulatory region of the biofilm formation modulating gene of interest.

33. The method of claim 32 wherein the biofilm formation modulating gene of interest is at least one of uvrY, csrA, sdiA, csrB, and barA.

34. An inhibitor of CsrA expression, comprising an isolated nucleotide sequence containing two or more repeats of the sequence element CAGGAUG.

35. A stimulator of biofilm formation, comprising an isolated sequence nucleotide containing two or more repeats of the sequence element CAGGAUG.

36. A modulator of biofilm formation, comprising an isolated amino acid sequence selected from a portion of the amino acid sequence of CsrA.

37. A method of modulating glycogen biosynthesis and catabolism in a bacterial strain comprising modulating the level or activity of CsrA.

38. The method of claim 37 wherein the strain is an E. coli strain, Salmonella strain, or a related gamma proteobacteria.

39. A method of modulating glycogen biosynthesis in a biofilm-forming bacterial strain, comprising inducing the presence within the bacterial strain of a nucleotide having two or more repeats of the sequence CAGGAUG.

40. Use of an inhibitor of biofilm formation in improving recovery of a mammalian patient suffering from infection by bacteria forming biofilm.

41. Use of claim 40 wherein the inhibitor of biofilm formation is an inhibitor of csrB transcription.

42. Use of claim 40 wherein the bacteria is an E. coli bacteria, Salmonella bacteria, or a related gamma proteobacteria.

43. Use of claim 40 wherein the patient is a human or domestic mammal.

44. A modulator of biofilm formation in a bacterial strain, comprising an agent having an AHL binding domain and a DNA binding domain and having at least 50% of the activity of sdiA in stimulating UvrA under physiological conditions.


SEQUENCE LISTING (8 sequences)

SEQ ID NO: 1 - RNA, 7 nt, Escherichia coli
caggaug

SEQ ID NO: 2 - RNA, 19 nt, Escherichia coli
  misc_feature (10)..(10): n is u, c, a, or g
ugcacacrrnyygugugug

SEQ ID NO: 3 - RNA, 19 nt, Escherichia coli
  misc_feature (10)..(10): n is u, c, a, or g
ugcacacyynrrgugugug

SEQ ID NO: 4 - RNA, 19 nt, Escherichia coli
ugcacacggauugugugug

SEQ ID NO: 5 - DNA, 28 bp, Escherichia coli
gagaatgcatacgccaaaatgaggacag

SEQ ID NO: 6 - DNA, 28 bp, Escherichia coli
gcggatccactcgacaagacatccatta

SEQ ID NO: 7 - DNA, 27 bp, Escherichia coli
cagcatcgctttcaggcaggagacttc

SEQ ID NO: 8 - DNA, 28 bp, Escherichia coli
cagttcgtggtcatcaacaagtagaacg